(19) United States
(12) Patent Application Publication
(10) Pub. No.: US 2010/0087706 A1
(43) Pub. Date: Apr. 8, 2010

(54) Lead Access

(75) Inventors: Baber Syed, Palo Alto, CA (US); Kurt Sparks, San Carlos, CA (US); Ken Wong, Saratoga, CA (US)

Correspondence Address: IntraPace, Inc. & Townsend & Townsend & Crew LLP, Two Embarcadero Center, Eighth Floor, San Francisco, CA, 94111-3834, US

(73) Assignee: IntraPace, Inc., Mountain View, CA (US)

(21) Appl. No.: 12/568,920

(22) Filed: Sep. 29, 2009

Related U.S. Application Data
(60) Provisional application No. 61/101,225, filed on Sep. 30, 2008.

(51) Int. Cl. A61B 1/00; A61B 18/18; A61B 17/34; A61B 17/94

(52) U.S. Cl. 600/104; 606/33; 606/185; 600/160

(57) ABSTRACT

The present invention discloses various methods and systems for positioning a guide wire between a patient's mouth and a skin site via an implant tract in a stomach. The method includes locating the desired tissue site in the stomach, such as with an endoscope or other suitable instrument. In some embodiments the desired tissue site is marked inside the stomach with a visible dye or light visible from the peritoneal cavity. An implant tract is created through the stomach wall at the desired tissue site, either from “inside-out” or “outside-in”. The implant tract may be made using a RF catheter, RF guide wire, an endoneedle, or other suitable instrument. The size of the implant tract depends on the device to be placed there, such as a stimulation lead. Diameter sizes of the tract may vary from 0.014″ to 0.250″. An access hole or access port is created at a skin site, using a Verres needle, RF catheter, RF guide wire, an endoneedle, or other suitable instrument. A guide wire is then positioned through the implant tract, access port and mouth, such that the guide wire extends between the mouth and the skin site access port via the implant tract in the stomach.

### CROSS-REFERENCES TO RELATED APPLICATIONS

The present application claims the benefit under 35 USC 119(e) of U.S. Provisional Application No. 61/101,225 filed Sep. 30, 2008; the full disclosure of which is incorporated herein by reference in its entirety.

### BACKGROUND OF THE INVENTION

1. Field of Invention

This invention relates to medical devices, systems, and methods. In exemplary embodiments, the invention provides a system and method for providing an implant tract through a desired tissue site in a stomach, such as a pes anserinus (“PES”) site, and positioning a guide wire between the implant tract and an external body site.

2. Background

Electrical stimulation of the gastrointestinal tract has been proposed to treat motility related disorders and other gastrointestinal diseases. The electrical stimulation has been proposed in a number of forms, such as pacing, electrical contractile stimulation or other stimulation, to treat various diseases or symptoms, such as nausea or obesity. Electrical stimulation has also been proposed to treat obesity by altering gastric motility, or by stimulating neural pathways. For example, one treatment method causes the stomach to retain food for a greater duration. Electrical stimulation has also been proposed to slow the gastric emptying to treat a disorder known as dumping syndrome where the stomach empties at an abnormally high rate into the small intestine causing various gastrointestinal disorders.

An early attempt at a gastric stimulation device included an electrode at the end of a nasogastric tube or catheter. The nasogastric tube was passed into the stomach transnasally.

Electrical stimulation was applied using an external stimulator unit through the electrode on the end of the tube. The return electrode was placed on the abdomen. This device required a transnasal procedure whenever stimulation was required.

Endoscopic devices have been disclosed for gastric stimulation, see for example related U.S. Pat. No. 6,535,764, fully incorporated herein by reference. U.S. Pat. No. 6,535,764 describes a gastric stimulator that is implanted by delivering the device through the esophagus of a subject and attaching to the stomach wall from the inside of the stomach.

Other devices used to pace or electrically stimulate the stomach have generally been implanted by accessing the outside of the stomach through an opening in the abdomen, either through open surgery or laparoscopic surgery. For example, electrodes have been attached to the stomach wall with attached leads extending through the abdomen. The leads are connected with a pacemaker device which is implanted in a subcutaneous or sub-muscular pocket at a remote location.

Improved systems and methods of accessing an implantation site in the stomach would be desirable. Such systems and methods should be easily performed, suitable for long term use, safe, and effective in treating the disorder or symptom, to name a few. In particular, such methods should be particularly suitable for treatment of obese patients who may have particular needs and limitations due to their condition. At least some of these objectives will be met by the present invention.

It would be desirable to provide improved methods for accessing an implantation site in the gastrointestinal tract, in particular the stomach, provide a guide wire from the implantation site to an external site that is compatible with a stimulation device lead, and provide an opening at the implantation site for secure attachment of the stimulation device lead to the organ wall.

### BRIEF SUMMARY OF THE INVENTION

The present invention discloses various methods for positioning a guide wire between a patient's mouth and a skin site via an implant tract at a desired tissue site in a stomach. In one embodiment, the desired tissue site is the pes anserinus (“PES”). The method includes locating the desired tissue site in the stomach, such as with an endoscope or other suitable instrument. In some embodiments the desired tissue site is marked inside the stomach with a visible dye or light visible from the peritoneal cavity. An implant tract is created through the stomach wall at the desired tissue site, either from “inside-out” or “outside-in”. The implant tract may be made using a RF catheter, RF guide wire, an endoneedle, or other suitable instrument. The size of the implant tract depends on the device to be placed there, such as a stimulation lead. Diameter sizes of the tract may vary from 0.014″ to 0.250″. An access hole or access port is created at a skin site, using a Verres needle, RF catheter, RF guide wire, an endoneedle, or other suitable instrument. A guide wire is then positioned through the implant tract, access port and mouth, such that the guide wire extends between the mouth and the skin site access port via the implant tract in the stomach.

### BRIEF DESCRIPTION OF THE DRAWINGS

FIG. 1 shows the location of the desired tissue site within the stomach.

FIGS. 2A and 2B show one embodiment of the invention (Method 1) using an “outside-in” approach to create an implant tract at the desired tissue site and positioning a guide wire using an endoscope on the stomach side and a percutaneous scope through the peritoneal side.

FIGS. 3A and 3B show one embodiment of the invention (Method 2) using an “inside-out” approach to create an implant tract at the desired tissue site and positioning a guide wire and a balloon flag using an endoscope through the stomach side and a percutaneous scope through the peritoneal side.

FIGS. 4A, 4B and 4C show one embodiment of the invention (Method 3) using an “outside-in” approach to create an implant tract at the desired tissue site and positioning dual guide wires using a combination of Natural Orifice Translumenal Endoscopic Surgery (NOTES) from the stomach side and a percutaneous endoscopic gastrostomy (PEG) procedure from the peritoneal cavity side.

FIGS. 5A and 5B show another embodiment of the invention (Method 4) using an “inside-out” approach to create an implant tract at the desired tissue site and positioning a guide wire and a balloon flag using Natural Orifice Translumenal Endoscopic Surgery (NOTES) from the stomach side and a percutaneous scope through the peritoneal side.

FIGS. 6A and 6B show another embodiment of the invention (Method 5) using an “inside-out” approach to create an implant tract at the desired tissue site and positioning a guide wire using Natural Orifice Translumenal Endoscopic Surgery (NOTES).

FIGS. 7A and 7B show one embodiment of a viewing lens that may be affixed onto the distal end of a scope.

FIG. 8 shows another embodiment of a viewing lens that may be affixed onto the distal end of a scope.

FIGS. 9A and 9B show another embodiment of the invention (Method 6) using an “outside-out” approach to create an implant tract at the desired tissue site and positioning a guide wire using a combination of Natural Orifice Translumenal Endoscopic Surgery (NOTES) from the stomach side and a percutaneous endoscopic gastrostomy (PEG) procedure from the peritoneal cavity side along with two graspers.

FIG. 10 shows one embodiment of a handleless forceps.

### DETAILED DESCRIPTION OF THE INVENTION

The present invention is directed to locating and creating an implant tract at a desired tissue site in a stomach and the placement of a guide wire through the tract from the desired tissue site to an access port on the skin. Once the implant tract is made and the guide wire is in place, the guide wire may be use to implant a device at the desired tissue site, such as a stimulation lead. While there are many desired tissue sites to choose from in the stomach, the present invention is directed to locating and creating an implant tract at the Pes Anserinus (“PES”) site in a stomach. While the following description describes the location and access of the PES site, the same methods and systems apply to any desired tissue site within the stomach.

FIG. 1 shows the location of a desired implant site 10, for example the PES site, in a stomach 15 in relation to other visceral organs. Access from outside the stomach to the site 10 is difficult. The left lobe of the liver 20 covers the desired implant site 10. Even with the stomach 15 insufflated, the liver 20 is moved toward the head, the small bowel and transverse colon 25 may be moved toward the feet, and at a minimum, the greater curve of the stomach is exposed from under the liver, but the site is still covered.

The present invention must also overcome other anatomical drivers accessing the desired implant site 10. For example, light from an endoscope positioned within the stomach near the desired implant site 10 is not reliably visible through the outer skin and there is no direct or consistent vector from the site to the skin. Both of these difficulties would make a direct approach from the skin difficult to the desired implant site 10, for example, using a Percutaneous Endoscopic Gastrostomy (PEG) approach to the desired implant site 10. Also, nominal thickness of the “skin-to-peritoneum” is 4″-6″ and the omentum likely covers the desired implant site 10 and anterior stomach wall. Visceral (mesentery) fat can occupy similar volume as the small bowel and in obese people (BMI>55), respiration may be severely compromised if peritoneal insufflation is employed to allow direct visualization of the desired implant site 10.

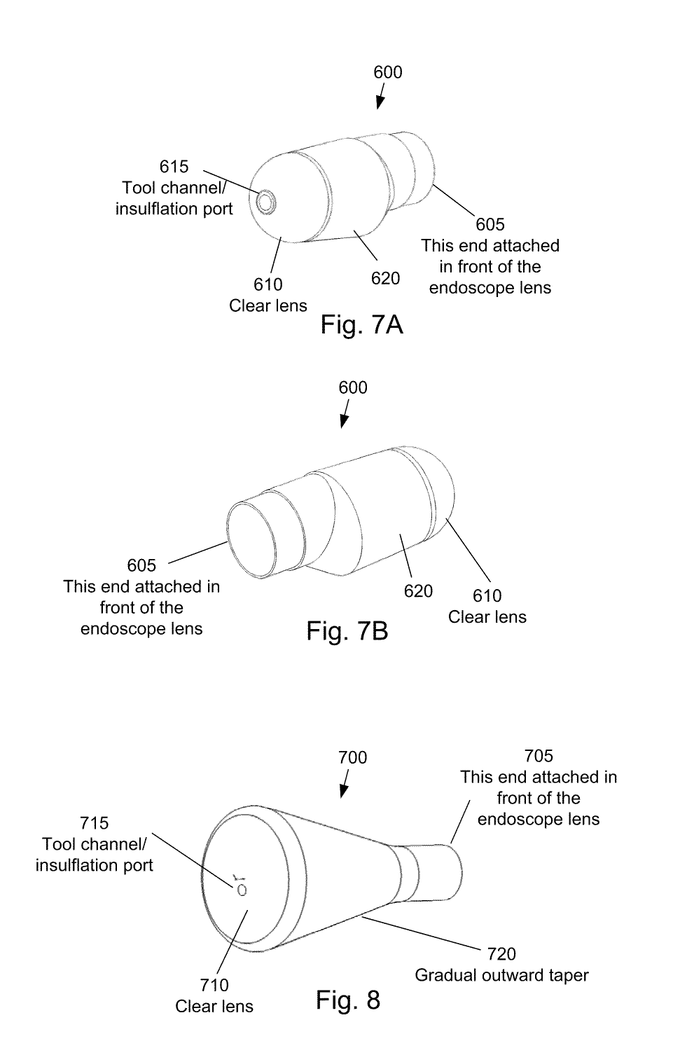

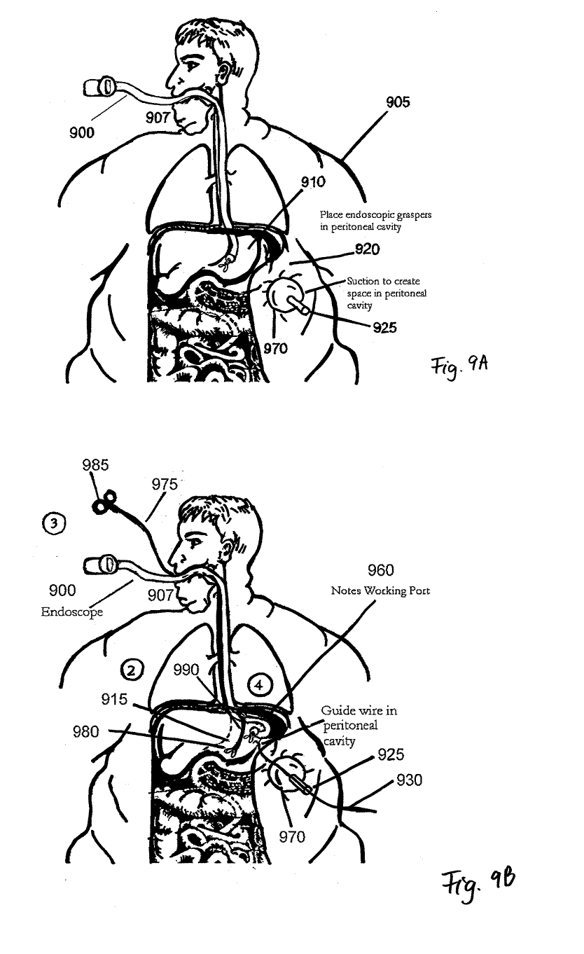

The present invention overcomes these problems by using both an internal (endoscopic) approach to the desired implant site 10 inside the stomach combined with an external approach in the peritoneal cavity using some form of peritoneal navigation to/from the desired implant site 10 to a skin port. A general method of peritoneal navigation includes introducing a scope that allows the operator to steer the scope within the peritoneal cavity. The instruments used in the procedures must be able to form a pathway around and/or through visceral fat and omentum, and be able to navigate between the liver, small bowel and transverse colon, with stomach insufflation titrated as required. For better viewing, some embodiments may use a viewing lens affixed onto the distal end of a viewing scope lens or laparoscope camera lens (See FIGS. 7A, 7B and 8). Another purpose of the viewing lens is to maintain the visceral organs a fixed distance from the viewing scope lens or laparoscope camera lens.
Desired procedural drivers of the present invention include:
No general anesthesia.
Prohibit or minimize inflation of the peritoneal cavity.
Titration of stomach insufflation as required.
Overtube, lavage (anti-microbial) treatment of stomach
Prohibit or minimize the use of fluoroscopy
Some methods require only one operator vs. two operators

The present invention is designed to create an implant tract at the desired implant site and place a guide wire from the implant tract to the skin. The implant tract can be made in the stomach wall from either the “outside-in” (from the peritoneal cavity into the stomach) or from the “inside-out” (from the stomach into the peritoneal cavity). Each of these methods is disclosed below. One of the objectives of these approaches is to make the implant tract size as small as possible.

Some of the embodiments disclosed use laparoscopic surgery to access the peritoneal cavity. There are a number of advantages to the patient using laparoscopic surgery versus an open surgical procedure. These include:
reduced blood loss, which equals less risk of needing a blood transfusion.
smaller incision, which equals less pain and shorter recovery time.
less pain, which equals less pain medication needed.
Although procedure times are usually slightly longer, hospital stay is less, and often with a same day discharge which equals a faster return to everyday living.
Reduced exposure of internal organs to possible external contaminants thereby reduced risk of acquiring infections.

In many of the embodiments disclosed a flexible endoscope is used with various instruments, as will be described in more detail below. The flexible endoscope is used to locate the desired tissue site within the stomach. The flexible endoscope may be of the type that is typically used by gastroenterologists in treating the upper gastrointestinal tract and in accessing the esophagus or stomach. The endoscope allows the physician to visualize while performing procedures on the upper gastrointestinal tract. The flexible endoscope may be, for example, a flexible fiber optic endoscope utilizing optic fibers for imaging. Such endoscopes typically include a fiber optic light guide and a complex objective lens at the distal end to focus the image. Alternatively, newer generation endoscopes utilize a charge coupled device (CCD) mounted at the distal end of the endoscope to generate images.

The endoscope comprises an elongate tube having a proximal handle portion and a distal portion. The endoscope may include a plurality of channels, such as an instrument channel. The instrument channel extends through the endoscope and provides an opening through which surgical instruments may be inserted to reach the site. Other instruments described with respect to the various embodiments herein may be introduced through the instrument channel, through an opening in an overtube, or alternatively, the instrument may be inserted along side of the endoscope, for example in an attached guide or sheath. Fiber optic light sources for illuminating the stomach extend through a fiber optic channel. A video lens may be located at the distal end of the endoscope, for receiving and focusing the image that is transmitted back through a channel in the endoscope. The endoscope may also include knobs coupled at the proximal handle for left/right and up/down steering mechanisms that are used to steer the distal portion of the endoscope in a manner that is generally known to one of ordinary skill in the art.

During procedures requiring an endoscope, or other instruments delivered through the mouth, the patient may be given a numbing agent that helps to prevent gagging. The endoscope is then passed through the mouth, pharynx, into the esophagus and into the stomach. If desired, an overtube may be used to protect the esophagus, which may become irritated with repeated insertion and removal of instruments. The overtube may also help prevent instruments and devices from inadvertently dropping into the trachea. In addition, an overtube may serve to protect the tools from the bacteria in the mouth and esophagus so that such bacteria are not passed on to the stomach wall. The overtube may also include additional channels for inserting additional instruments. As an alternative to an overtube, additional instruments may be attached to the outside of the endoscope and inserted through the esophagus. Preferably the instruments inserted into the patient's stomach are coated with an antibacterial material, in particular, the instruments that are used to pierce or otherwise come in contact with the stomach wall. Exemplary embodiments of endoscopic delivery systems and endoscopically delivered stimulation devices and systems are described in U.S. Pat. No. 6,535,764, incorporated herein by reference for all purposes.

In some embodiments, a balloon catheter may be used, such as a percutaneous transluminal angioplasty (PTA) catheter or balloon catheter. The balloon is located at a distal end of a shaft, coupled to an inflation lumen and catheter may range in size from 5 mm 75 mm to create space within the peritoneal cavity. In some embodiments the shaft is flexible while in other embodiments the shaft may be made of ridged materials such as steel spring or wire to improve its ability to steer. The balloon catheter may be steerable in a manner similar to a guide wire to allow it to be ideally positioned such that a tool from an endoscope or laparoscope is able to grasp the balloon. In some embodiments, the balloon may be inflated with enough pressure to dilate an opening in the stomach wall, such as a working port discussed below. In other embodiments, the balloon is inflated within the peritoneal cavity to serve as a “balloon flag” viewable from outside the stomach and may be made of a specific color or highly visible material. The balloon may be formed of either a compliant or non-compliant material such as, e.g., polyurethane, polyethylene, polyester or a rubber material such as silicone, depending on the use of the catheter.

The following methods disclosed may be viewed as two basic types of procedures. The first is the combination of an endoscope within the stomach and percutaneous scope in the peritoneal cavity. The second combines Natural Orifice Translumenal Endoscopic Surgery (NOTES) from within the stomach into the peritoneal cavity.

In general, the present invention discloses various methods for positioning a guide wire between a patient's mouth and a skin site via an implant tract in a stomach. The method includes locating the desired tissue site in the stomach, such as with an endoscope or other suitable instrument. In some embodiments the desired tissue site is marked inside the stomach with a visible dye or light visible from the peritoneal cavity. An implant tract is created through the stomach wall at the desired tissue site, either from “inside-out” or “outside-in”. The implant tract may be made using a RF catheter, RF guide wire, an endoneedle, or other suitable instrument. The size of the implant tract depends on the device to be placed there, such as a stimulation lead, or the size of the instrument used to create the tract. Diameter sizes of the tract may vary from 0.014″ to 0.250″. An access hole or access port is created at a skin site, using a Verres needle, RF catheter, RF guide wire, or other suitable instrument. A guide wire is then positioned through the implant tract, access port and mouth, such that the guide wire extends between the mouth and the skin site access port via the implant tract in the stomach. The examples disclosed below show five methods for creating an implant tract and placing a guide wire through the tract to an access port. The disclosed methods are shown as examples, as other combinations of devices may be combined to accomplish the same outcome.

### Method 1—Endoscopic+Percutaneous Scope (Outside-In)

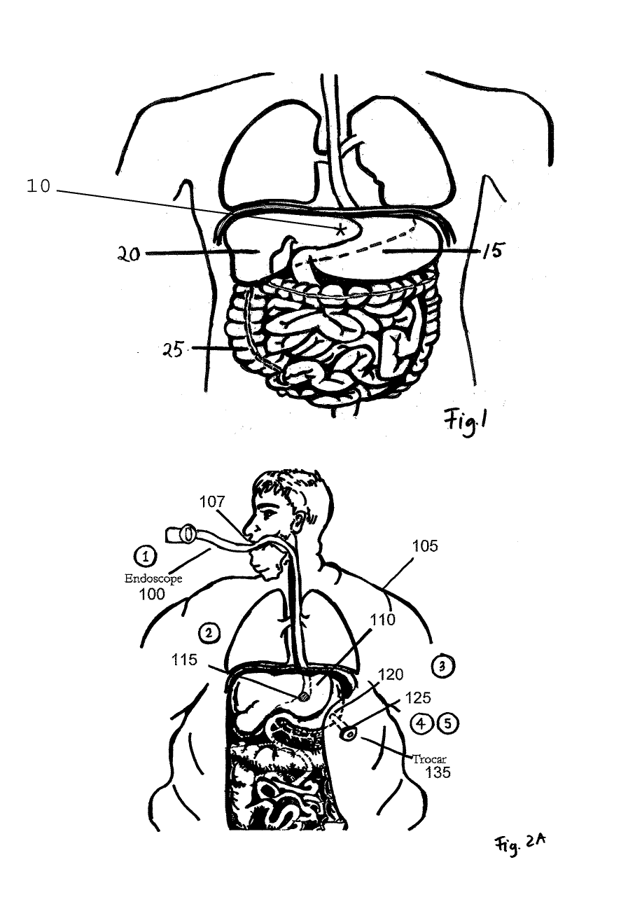

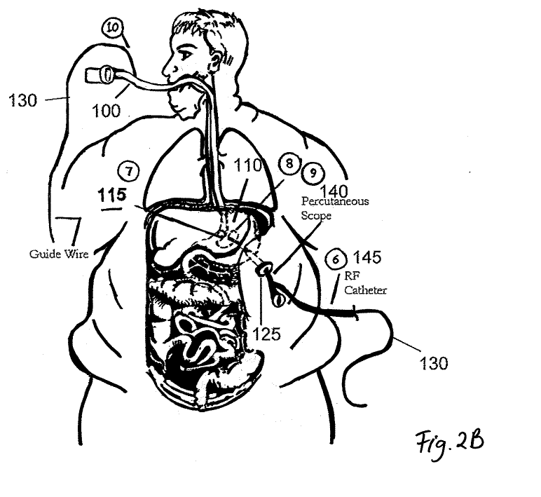

FIGS. 2A and 2B show one embodiment of the invention using an “outside-in” approach to create an implant tract at the desired tissue site and positioning a guide wire. Method 1 uses an endoscope on the stomach side and a percutaneous scope through the peritoneal side. Some of the equipment used in this embodiment includes a scope navigatable in the peritoneal cavity (peritoneal scope), endoscope, Verres needle, dilator & trocar, guide wire, peritoneal scope hood or viewing lens and RF catheter.

Method 1 includes the following steps (see FIG. 2A for steps 1-5 and FIG. 2B for steps 6-10):
1. Place the endoscope 100 into the mouth of a patient 105 until it is inside the stomach lumen 110.
2. Locate the desired tissue site 115 with the endoscope 100 and mark the site 115 with a visible dye, visible from the peritoneal cavity 120. The site may also be a light visible from the peritoneal cavity through the stomach wall.
3. Enter the peritoneal cavity 120 through a skin site with the Verres needle.
4. Advance the 0.035″ guide wire 130 into the peritoneal cavity and dilate the skin site.
5. Place a trocar 135 in the skin opening and remove the 0.035″ guide wire creating an access port 125 at the skin site.
6. Insert the peritoneal scope 140 with the viewing lens 600 and RF catheter 145 into the peritoneal cavity 120 through the access port 125.
7. Navigate the percutaneous scope 140 with the RF catheter 145 to the desired tissue site 115 through the peritoneal cavity 120 and view the visible dye or light at the desired tissue site 115 with the percutaneous scope. The peritoneal scope hood or viewing lens 600 is designed to visualize around the visceral organs and steer and advance the scope in the peritoneal cavity without the need for peritoneal insufflation. Alternatively, titration and cycling of peritoneal insufflation could be performed as required to assist in providing enhanced viewing during the procedure.
8. Ablate an implant tract through the desired tissue site 115 into stomach lumen using the RF catheter 145. The size of the implant tract may range from 0.020″ to 0.060″ but is not so limited.
9. Advance the 0.035″ guide wire 130 from the RF catheter 145 and into the lumen of the stomach 110.
10. Grab the guide wire 130 in the stomach 110 with the endoscope 100 and retract the guide wire 130 out of the mouth 107. The guide wire 130 is now positioned between the mouth 107 and skin access port 125 via the implant tract 115.

Some of the advantages of Method 1 include:
Improved visibility and indicators from two scopes from both directions.
Guide wire is easily captured in the lumen of the stomach and withdrawn from the mouth.
Small implant tract from RF catheter.
Overtube not required.
Fluoroscopic view not required.

One disadvantage is the procedure involves two trained physicians working simultaneously with the two scopes.

Method 2—Endoscope+Percutaneous Scope with Balloon Flag (Inside-Out)

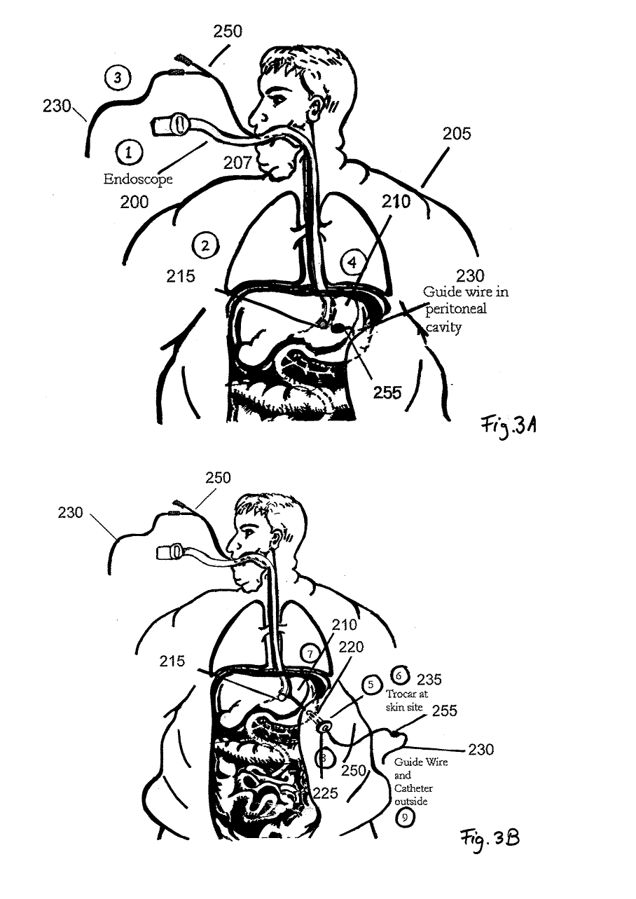

FIGS. 3A and 3B show one embodiment of the invention using an “inside-out” approach to create an implant tract at the desired tissue site and positioning a guide wire. Method two uses an endoscope through the stomach side and a percutaneous scope through the peritoneal side. Some of the equipment used in this embodiment include a peritoneal scope, endoscope, fluoroscope, Verres needle, balloon catheter, dilator & trocar, guide wire and peritoneal scope hood viewing lens.

Method 2 includes the following steps (see FIG. 3A for steps 1-4 and FIG. 3B for steps 5-9):
1. Place the endoscope 200 into the mouth 207 of a patient 205 until it is inside the stomach lumen 210.
2. Locate the desired tissue site 215 with the endoscope 200 and create an implant tract 215 with a 0.035″ guide wire 230 across the stomach wall at the desired tissue site using the endoneedle.
3. Remove the endoscope 200 and position it in the stomach lumen 210 next to the 0.035″ guide wire 230.
4. Place the balloon catheter 250 over the guide wire 230 and across the stomach wall through the implant tract 215. Inflate a balloon on the balloon catheter 250 within the peritoneal cavity to create a balloon flag 255.
5. Puncture the skin site with a Verres needle and dilate the site creating an access port 225 at the skin site.
6. Place a trocar 235 in the access port and insert the peritoneal scope with the scope hood or viewing lens into the peritoneal cavity 220.
7. Navigate the peritoneal scope to the implant tract 215 through the peritoneal cavity 220 and view the balloon flag 255. Grab the balloon flag 255 and/or catheter 250 with graspers.
8. Pull the peritoneal scope and the catheter 250 through the access port 225 at the skin site.
9. Push the guide wire 230 out of the catheter 250. The guide wire 230 is now positioned between the mouth 207 and skin access port 225 via the implant tract 215.

Some advantages of Method 2 include:
Improved visibility and indicators from two scopes.
Large balloon in the peritoneal cavity may help in pushing the liver out of the way.
Procedure may involve a single physician and a trained technician.
Small implant tract size (0.100″).

Some disadvantages of Method 2 include:
Fluoroscope may be required to place the guide wire in the peritoneal cavity.
Overtube and “antimicrobial lavage” may be utilized to decrease gastric bioburden prior to accessing the peritoneal cavity from the gastric lumen.

### Method 3—NOTES+Dual Guide Wire (Outside-In)

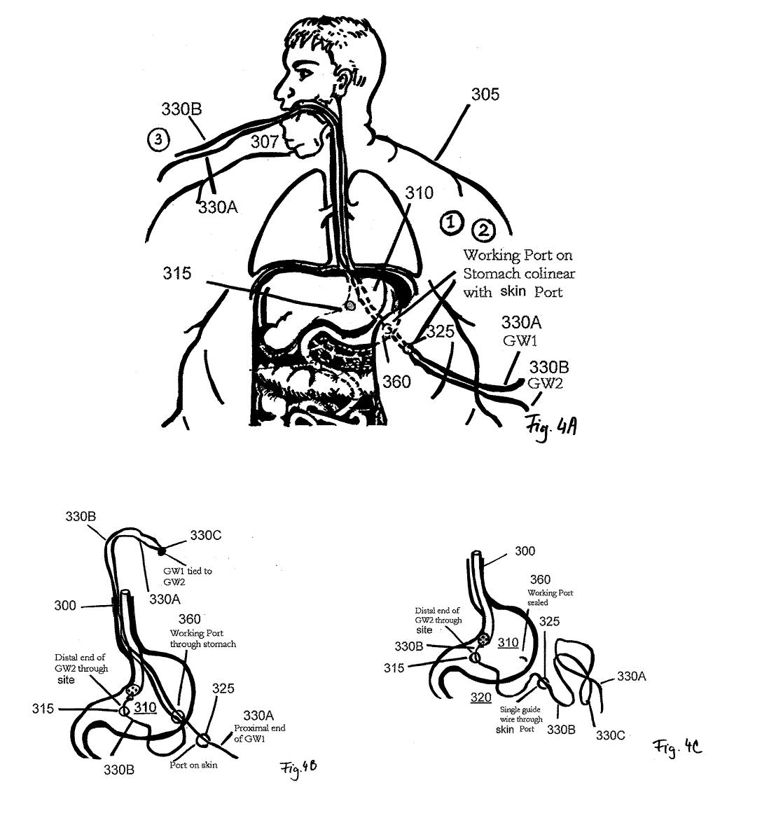

FIGS. 4A, 4B and 4C show one embodiment of the invention using an “outside-in” approach to create an implant tract at the desired tissue site and positioning a guide wire using a combination of Natural Orifice Translumenal Endoscopic Surgery (NOTES) from the stomach side and a percutaneous endoscopic gastrostomy (PEG) procedure from the peritoneal cavity side. Some of the equipment used in this embodiment includes an endoscope, PEG needle, endoscope viewing lens or scope hood, RF catheter and custom guide wires.

Method 3 includes the following steps (see FIG. 4A for steps 1-5 and FIGS. 4B and 4C for steps 6-10):
1. Using a PEG needle create an access port 325 in the skin and a “working port” 360 in the stomach.
2. Feed in two guide wires 330A, 330B, through the access port 325 and working port 360 into the stomach 310.
3. Using the endoscope 300, retract both guide wires out of the mouth.
4. Track the endoscope and a percutaneous transluminal angioplasty (PTA) catheter over guide wire 330B into the stomach. Dilate the working port 360 to make it larger to fit the endoscope and advance the endoscope into peritoneal cavity 320 (may be performed with a Novare instrument).
5. Remove the PTA catheter and load the RF catheter into the endoscope. Pull back on guide wire 330B so that the end is within the endoscope and RF catheter, and retroflex the endoscope to the desired tissue site 315. To facilitate steering the endoscope to the desired tissue site, air may be titrated through the endoscope and into the working space in the peritoneal cavity in the proximity of the desired tissue site.
6. Ablate an implant tract through the stomach wall at the desired tissue site into stomach lumen using the RF catheter. For example, the implant tract may be 0.060″.
7. Advance guide wire 330B into the stomach 310 through the end of the endoscope.
8. Retract the endoscope 300 from patient and re-insert it into stomach to grasp end of guide wire #2.

9. Connect “mouth-ends” 330C of guide wire 330A and guide wire 330B together.
10. Retract guide wire 330A fully from the access port 320 through the working port 360, pulling proximal end of guide wire 330B (still connected to guide wire 330A) out through the access port 325. Guide wire 330B is now positioned between the mouth 307 and skin access port 325 via the implant tract 315. The working port 360 may be closed by known means.

Some of the advantages of Method 3 include:
Small implant tract from RF catheter (0.060″).
Procedure may involve a single physician and a trained technician.
Fluoroscope not required.

Some disadvantages of Method 3 include:
First must assume successful PEG needle placement between the skin and stomach.
Overtube may be required to maintain aseptic condition.

### Method 4—NOTES+Balloon Flag (Inside-Out)

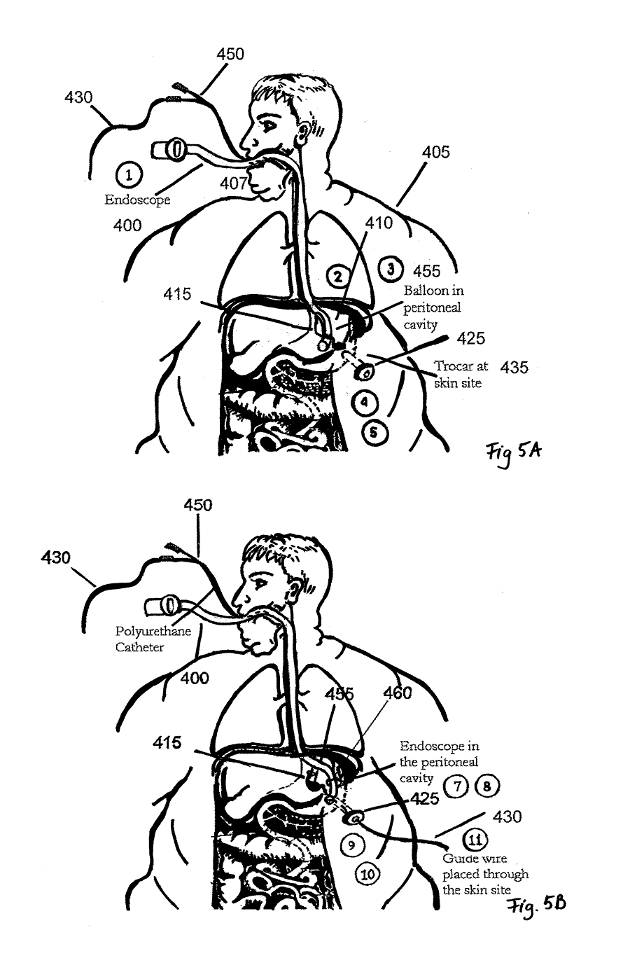

FIGS. 5A and 5B show another embodiment of the invention using an “inside-out” approach to create an implant tract at the desired tissue site and positioning a guide wire using Natural Orifice Translumenal Endoscopic Surgery (NOTES) and the balloon flag procedure discussed above. Some of the equipment used in this embodiment includes a endoscope, Verres needle, compliant balloon catheter, guide wire, fluoroscope, dilator & trocar, peritoneal scope viewing lens, and RF catheter.

Method 4 includes the following steps (see FIG. 5A for steps 1-6 and FIG. 5B for steps 7-11):
1. Place overtube into the mouth 407 of a patient 405 and then insert the endoscope 400 through the overtube until it is inside the stomach lumen 410, lavage the stomach.
2. Locate the desired tissue site 415 with the endoscope and create an implant tract 415 with a 0.035″ guide wire across the desired tissue site using the endoneedle. Place the balloon catheter 450 over the guide wire 430 and across the stomach wall through the implant tract 415. Inflate the balloon catheter 455 within the peritoneal cavity to create a balloon flag.
3. Remove the endoscope 400.
4. Puncture the skin site with a Verres needle and dilate the site creating an access port 425 at the skin site.
5. Place a trocar 435 in the access port.
6. Re-introduce the endoscope 400 into the stomach and cross a fundus site to create a “working port” 460 with the endoneedle and a 0.035″ guide wire. Feed the guide wire into the peritoneal cavity using fluoroscopy. Remove the endoneedle.
7. Advance the PTA catheter over guide wire and dilate the fundus working port. Advance the endoscope 400 into peritoneal cavity 420.
8. Remove the PTA catheter.
9. Advance the endoscopic grabber into the tool channel.
10. Navigate the endoscope 400 to the implant site 415 through the peritoneal cavity 420 and locate the balloon flag 455. Grab the balloon flag 455 with the grabber and drag the balloon to the skin access port 425 at the skin site.
11. Feed the guide wire 430 from the catheter into skin access port 425 and exit proximal end of the skin access port 425. The guide wire 430 is now positioned between the mouth 407 and skin access port 425 via the implant tract 415. Remove the balloon catheter.

Some advantages of Method 4 include:
Small implant tract from endoneedle puncture (0.100″).
Single operator.

Some disadvantages of Method 4 include:
Overtube, lavage may be required.
Fluoroscope or other remote imaging may be required to place the guide wire in the peritoneal cavity.

### Method 5—NOTES+5 mm Endoscope (Inside-Out)

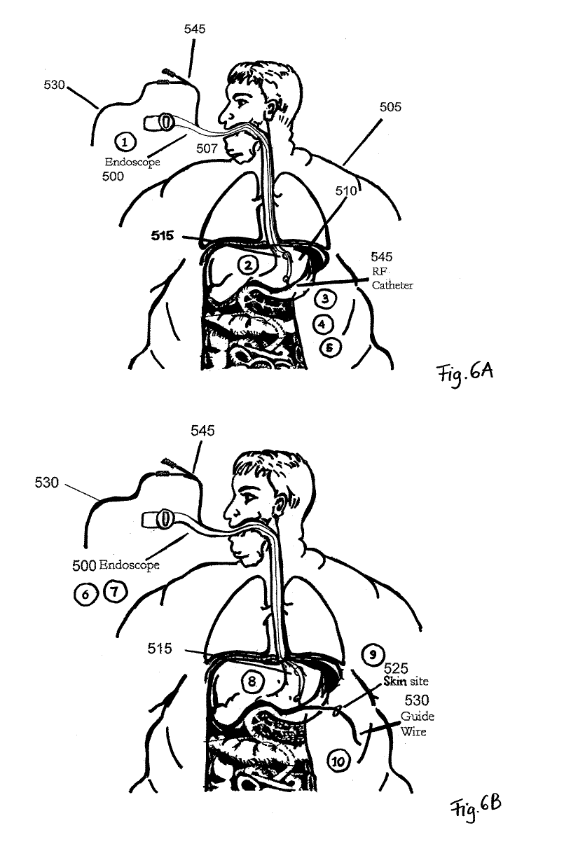

FIGS. 6A and 6B show another embodiment of the invention using an “inside-out” approach to create an implant tract at the desired tissue site using Natural Orifice Translumenal Endoscopic Surgery (NOTES). Some of the equipment used in this embodiment includes a 5 mm endoscope, Verres needle, balloon catheter, guide wire, fluoroscope, dilator & trocar, scope viewing lens, and RF catheter.

Method 5 includes the following steps (see FIG. 6A for steps 1-5 and FIG. 6B for steps 6-10):
1. Place overtube and endoscope, lavage.
2. Affix viewing lens 600 to the endoscope 500. Place the endoscope 500 and endoneedle into the mouth 507 of a patient 505 and advance them inside the stomach lumen 510.
3. Locate the desired tissue site with the endoscope 500 and cross the PES with the endoneedle creating an implant tract 515. Advance the 0.035″ guide wire 530 through the implant tract 515 into the peritoneal cavity. Fluoroscopy may be employed to assist in advancing the guide wire into the peritoneal cavity. Remove the endoneedle.
4. Advance a 5 mm PTA catheter over guide wire into the implant tract and dilate the implant tract 515 with a balloon.
5. Advance the endoscope 500 into the peritoneal cavity 520 through the dilated implant tract 515.
6. Remove the PTA catheter.
7. Advance the RF catheter 545 into the endoscope tool channel.
8. Navigate the endoscope 500 through the peritoneal cavity 520 to the peritoneal wall.
9. Advance the RF catheter 545 through abdominal wall, subcutaneous fat and skin using ablation to create an access port 525 at the skin site.
10. Once through skin access port 525, advance the 0.035″ guide wire 530 through the access port 525. Retract/remove the endoscope and RF catheter. The guide wire 530 is now positioned between the mouth 507 and skin access port 525 via the implant tract 515.

One advantage of Method 5 is the need for only a single operator.

Some disadvantages of Method 5 include:
Overtube may be required
Fluoroscope may be required to place the guide wire in the peritoneal cavity
Large implant tract (0.200″)
   Method 6—NOTES with Two Graspers (Outside-in)

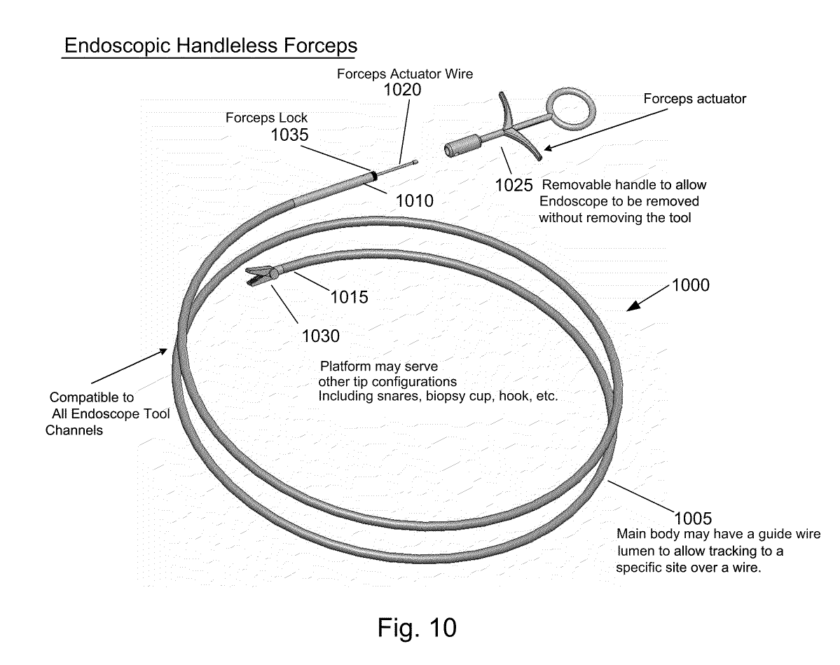

FIGS. 9A and 9B show one embodiment of the invention using an “outside-in” approach to create an implant tract at the desired tissue site and positioning a guide wire using the Handleless Forceps (see FIG. 10) and a combination of Natural orifice Translumenal Endoscopic Surgery (NOTES) from the stomach side and a percutaneous endoscopic gastrotomy (PEG) procedure from the peritoneal cavity side. Some of the equipment used in this embodiment includes an endoscope, endoneedle, PEG needle, LapCap™, endoscope viewing lens or scope hood, balloon catheter and guide wires.

Method 6 includes the following steps (see FIG. 9A and FIG. 9B):
1. Use a PEG needle to create an access port 925 at a skin site and a “working port” 960 in the stomach 910 with the aid of a suctioning device 970 such as a LapCap™ to create space within the peritoneal cavity 920.
2. Feed a guide wire 930 through the access port 925 and into the stomach 910.
3. Place an endoscope 900 in to the stomach 910 and dilate an implant tract 915 at a desired site using an endoneedle, a 2^{nd} guide wire, and a balloon.
4. Remove the balloon and track a handleless endoscopic forceps 975 over the guide wire through the mouth 907 and place a distal end 980 in the peritoneal cavity 920 via the implant tract 915. Remove a handle 985 from the handleless forceps 975 and retract the endoscope 900 and 2^{nd} guide wire out of the mouth 907. Replace the handle 985 on to the forceps 975.
5. Replace the endoscope 900 in the stomach 910 and retract the guide wire 930 placed via the PEG site 925 through the tool channel of the endoscope 900 and out of the mouth 907. Place a catheter over the guide wire and dilate the working port 960 to allow the endoscope 900 to go in to the peritoneum 910 through the working port.
6. Remove the balloon catheter, grab the guide wire 930 using an endoscopic grasper 990 and push the guide wire 930 back in to the peritoneum.
7. Steer the endoscope 900 towards the implant tract 915 while grasping the guide wire 930. Position the guide wire 930 into the jaws 980 of the handleless forceps 975 and lock the forceps.
8. Pull the guide wire 930 through the mouth 907 via the implant tract 915 by retracting the handleless forceps 975 completely out of the mouth.
9. The guide wire 930 is now placed from the skin site 925 to the mouth 907 via the implant tract 915. The working port 960 may be closed by known means.

Some of the advantages of Method 6 include:
Small implant tract from endoneedle puncture (0.100″).
Single operator.

Some disadvantages of Method 6 include:
Overtube may be required
Fluoroscope required to place the guide wire in the peritoneal cavity

Table 1 below shows a summary of the attributes of the methods described.

| METHOD | | Attributes | | | |
|---|---|---|---|---|---|
| Procedure Type | | Overtube | Tract (in) | # of operators | Fluoroscope |
| 1. | Endoscope + Percutaneous Scope | No | 0.060 | Two Physicians | No |
| 2. | Endoscope + Percutaneous Scope | Yes | 0.100 | One Physician | Yes |
| | with Balloon Flag | | | One Technician | |
| 3. | NOTES + Dual Guide Wires | Yes | 0.060 | One Physician | No |
| | | | | One Technician | |
| 4. | NOTES with Balloon Flag | Yes | 0.100 | One Physician | Yes |
| | | | | One Technician | |
| 5. | NOTES with 5 mm Endoscope | Yes | 0.200 | One Physician | Yes |
| | | | | One Technician | |
| 6. | NOTES with Two Graspers | Yes | 0.100 | One Physician | Yes |
| | | | | One Technician | |

FIGS. 7A and 7B show one embodiment of a viewing lens 600 having an attachment portion 605 configured to securely and sealingly attach to the distal end of an endoscope or a laparoscope (not shown). The viewing lens 600 includes a clear lens 610 so that the scope or camera can view out. The viewing lens 600 has length compatible with the focal length of the endoscope or laparoscope to allow tissue at a fixed distance to be in focus. The diameter of the viewing lens ranges from 5 mm-15 mm and the length may range from 0.1″−2.0″. The clear lens 610 is optically clear and may be made of a ridged material such as plastic, glass, or any combination of the two materials. A portion of the viewing lens or sides 620 may be opaque to enhance visibility within the peritoneal cavity. The viewing lens 600 may incorporate a port 615 such that it communicates directly with the endoscope's tool channel to allow the use of other tools such as an RF catheter or guide wire. The viewing lens 600 may incorporate a port 615 for insufflation and vacuum to allow titration of gas within the peritoneal cavity. Viewing lens 600 may have a spherical tip to prevent trauma to the surrounding organs.

FIG. 8 shows another embodiment of a viewing lens 700, similar to lens 600, having an attachment portion 705 configured to securely and sealingly attach to the distal end of an endoscope or a laparoscope (not shown). The viewing lens 700 includes a clear lens 710 that works in conjunction with the scope camera to increase the field of view, which may include an outward taper 720 along its length to improve visibility. The viewing lens 700 has length compatible with the focal length of the endoscope or laparoscope to allow tissue at a fixed distance to be in focus. The diameter of the viewing lens ranges from 5 mm-15 mm and the length may range from 0.1″−2.0″. The clear lens 710 is optically clear and may be made of a ridged material such as plastic, glass, or any combination of the two materials. The distal body of the viewing lens 700 may be made of optically clear pliable materials such as polyurethane or silicone rubber to allow the distal end to expand like a balloon. A portion of the viewing lens, such as the sides 720, may be opaque to enhance visibility within the peritoneal cavity. The viewing lens 700 may incorporate a port 715 such that it communicates directly with the scope's tool channel to allow the use of other tools such as an RF catheter or guide wire. The viewing lens 700 may incorporate a port 715 for insufflation and vacuum to allow titration of gas within the peritoneal cavity.

FIG. 10 shows one embodiment of a handleless forceps 1000 that may be used in one or more of the methods disclosed above. The handleless forceps 1000 includes a flexible body 1005 with a proximal end 1010 and a distal end 1015. The flexible body 1005 may be sized to fit within a guide catheter or a tool channel of an endoscope. The flexible body 1005 may also include a guide wire lumen to allow tracking to a specific site over a guide wire. An actuator wire 1020 extends through the body 1005 and is coupled to a removable handle 1025 near the proximal end and a forceps 1030 near the distal end 1015. The forceps 1030 may be locked with a forceps lock 1035 prior to removal of the handle. The removable handle 1025 allows the endoscope to be removed without removing the forceps 1030. Other tool configurations may be used in place of the forceps 1030, for example snares, biopsy cup, hook, or other suitable tools.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

What is claimed is:

1. A method for positioning a guide wire between a patient's mouth and a skin site via a desired tissue site in a stomach, the method comprising:
locating the desired tissue site in the stomach;
creating an implant tract at the desired tissue site;
creating an access port at the skin site; and
advancing a guide wire through the implant tract at the desired tissue site, the access port at the skin site and the mouth, wherein the guide wire extends between the mouth and the skin site via the implant tract.

2. The method of claim 1, wherein the desired tissue site is a pes anserinus (“PES”) site in the stomach.

3. The method of claim 1, wherein locating the desired tissue site in the stomach comprises advancing an endoscope orally into the stomach and viewing the desired tissue site with the endoscope.

4. The method of claim 1, wherein once the desired tissue site is located, the method further comprises visually marking the desired tissue site within the stomach.

5. The method of claim 1, wherein creating an implant tract at the desired tissue site comprises ablating the desired tissue site with an RF catheter from inside the stomach to outside the stomach.

6. The method of claim 1, wherein creating an implant tract at the desired tissue site comprises ablating the desired tissue site with an RF catheter from outside the stomach to inside the stomach.

7. The method of claim 1, wherein creating an implant tract at the desired tissue site comprises puncturing the desired tissue site with an endoneedle.

8. The method of claim 1, wherein creating an access port at the skin site comprises puncturing the skin site with the Verres needle.

9. The method of claim 1, wherein creating an access port at the skin site comprises a percutaneous endoscopic gastrostomy (PEG) port.

10. The method of claim 1, wherein creating an access port at the skin site comprises ablating the skin site using an RF catheter.

11. A method for placing a guide wire from a desired tissue site in a patient's stomach to a skin site, the method comprising:
visually marking the desired tissue site within the stomach;
accessing a peritoneal cavity through an access port at the skin site with a percutaneous scope with RF catheter;
advancing the percutaneous scope with RF catheter to the marked desired tissue site;
creating an implant tract at the desired tissue site from the peritoneal cavity into the stomach with the RF catheter; and
advancing a distal end of the guide wire through the percutaneous scope with RF catheter and into the stomach through the implant tract.

12. The method of claim 11, further comprising:
grabbing the distal end of the guide wire; and
extracting the distal end of the guide wire from the stomach through a mouth of the patient, wherein the guide wire extends between the mouth and the skin site via the implant tract.

13. The method of claim 11, wherein the desired tissue site is a pes anserinus (“PES”) site in the stomach.

14. The method of claim 11, further comprising locating the desired tissue site from within the stomach by advancing an endoscope orally into the stomach and viewing the desired tissue site with the endoscope.

15. The method of claim 11, wherein visually marking the desired tissue site comprises a dye visible from the peritoneal cavity.

16. The method of claim 11, wherein visually marking the desired tissue site comprises a light visible from the peritoneal cavity.

17. The method of claim 11, further comprising insufflating the peritoneal cavity while advancing the catheter to the desired tissue site.

18. A method for positioning a guide wire between a patient's mouth and a skin site via a desired tissue site in a stomach, the method comprising:
creating an implant tract at the desired tissue site;
advancing a balloon catheter orally into the stomach and through the implant tract;
inflating a balloon on the balloon catheter within the peritoneal cavity creating a balloon flag;
accessing the peritoneal cavity through an access port at the skin site;
grasping the balloon flag within the peritoneal cavity with an ENT scope;
extracting the balloon flag and catheter to the access port; and
advancing guide wire through the balloon catheter and out the access port at the skin site, wherein the guide wire extends between the mouth and the skin site via the implant tract.

19. The method of claim 18, wherein the desired tissue site is a pes anserinus (“PES”) site in the stomach.

20. The method of claim 18, further comprising locating the desired tissue site from within the stomach by advancing an endoscope orally into the stomach and viewing the desired tissue site with the endoscope.

21. The method of claim 18, further comprising creating an access port at the skin site by puncturing the skin site with the Verres needle.

22. The method of claim 18, wherein creating an implant tract at the desired tissue site comprises advancing a guide wire across the desired tissue site using an endoscopic needle.

23. A method for positioning a guide wire between a patient's mouth and a skin site via a desired tissue site in a stomach, the method comprising:
creating a working port into the stomach from an access port at the skin site;
advancing proximal ends of first and second guide wires through the access port and working port into the stomach and out of the patient's mouth;
advancing an RF catheter into the stomach and through the working port into the peritoneal cavity;
withdrawing the distal end of the second guide wire into the catheter;
advancing the RF catheter to the desired tissue site;
creating an implant tract at the desired tissue site with the RF catheter;
advancing the distal end of the second guide wire into the stomach and out of the mouth of the patient;
withdrawing the catheter out of the mouth;
securing the proximal ends of the first and second guide wires together; and
pulling a distal end of the first guide wire from the access port, thereby extracting the secured proximal ends of the first and second guide wires through the working port and out of the access port, wherein the second guide wire extends between the mouth and the skin site via the implant tract.

24. The method of claim 23, wherein the desired tissue site is a pes anserinus (“PES”) site in the stomach.

25. The method of claim 23, further comprising sealing the working port.

26. The method of claim 23, wherein the access port and working port are made using a percutaneous endoscopic gastrostomy (PEG) procedure.

27. A method for positioning a guide wire between a patient's mouth and a skin site via a desired tissue site in a stomach, the method comprising:
creating an implant tract at the desired tissue site from the stomach into a peritoneal cavity;
advancing a balloon catheter orally into the stomach, through the implant tract;
inflating a balloon on the balloon catheter within the peritoneal cavity creating a balloon flag;
creating a working port from the stomach to peritoneal cavity;
advancing an endoscope orally into the stomach, through the working port into the peritoneal cavity;
grabbing the balloon flag with an endoscopic grabber;
creating an access port at the skin site;
dragging the balloon flag and catheter to the access port; and
feeding a guide wire through the catheter and the access port; and
withdrawing the catheter and the endoscope; wherein the guide wire extends between the mouth and the skin site via the implant tract.

28. The method of claim 27, wherein the desired tissue site is a pes anserinus (“PES”) site in the stomach.

29. The method of claim 27, further comprising sealing the working port.

30. A method for positioning a guide wire between a patient's mouth and a skin site via a desired tissue site in a stomach, the method comprising:
creating an implant tract at the desired tissue site into a peritoneal cavity;
advancing an endoscope with an RF catheter orally into the stomach and through the implant tract;
navigating the endoscope through the peritoneal cavity;
creating an access port at the skin site using the RF catheter; and
advancing a guide wire through the endoscope and the access port, wherein the guide wire extends between the mouth and the skin site via the implant tract.

31. The method of claim 30, wherein the desired tissue site is a pes anserinus (“PES”) site in the stomach.

32. The method of claim 30, further comprising withdrawing the endoscope and RF catheter

33. A system for positioning a guide wire between a patient's mouth and a skin site via a desired tissue site in a patient's stomach comprising:
a marking device for marking the desired tissue site within the stomach;
a percutaneous scope with RF catheter configured to access a peritoneal cavity through an access port at the skin site, advance to the marked site, and create an implant tract into the stomach at the marked site;
a guide wire advanceable through the percutaneous scope with RF catheter into the stomach; and
an endoscope configured to retrieve the guide wire from the stomach, wherein the guide wire extends between the mouth and the skin site via the implant tract.

34. The system of claim 33, wherein the desired tissue site is a pes anserinus (“PES”) site in the stomach.

35. The system of claim 33, wherein the marking device comprises a dye visible from the peritoneal cavity.

36. The system of claim 33, wherein the marking device comprises a light visible from the peritoneal cavity.

37. A system for positioning a guide wire between a patient's mouth and a skin site via a desired tissue site in a stomach, the method comprising:
an endoscopic needle for creating an implant tract at the desired tissue site;
a balloon catheter configured to orally advance into the stomach and through the implant tract;
an inflation device configured to inflate a balloon on the balloon catheter within the peritoneal cavity creating a balloon flag;
an ENT scope configured to access the peritoneal cavity through an access port at the skin site, grasp the balloon flag within the peritoneal cavity and extract the balloon flag and catheter to the access port; and
a guide wire advanceable through the balloon catheter and out the access port at the skin site, wherein the guide wire extends between the mouth and the skin site via the implant tract.

38. The system of claim 37, wherein the desired tissue site is a pes anserinus (“PES”) site in the stomach.

39. The system of claim 37, further comprising an endoscope for locating the desired tissue site from within the stomach.

40. The system of claim 37, further comprising a Verres needle for creating an access port at the skin site.

41. A system for positioning a guide wire between a patient's mouth and a skin site via a desired tissue site in a stomach, the method comprising:
an endoscopic needle for creating an implant tract at the desired tissue site;
an endoscope with an RF catheter configured to orally advance into the stomach and through the implant tract, navigate through the peritoneal cavity and create an access port at the skin site using the RF catheter; and
guide wire advanceable through the endoscope and the access port, wherein the guide wire extends between the mouth and the skin site via the implant tract.

42. The system of claim 41, wherein the desired tissue site is a pes anserinus (“PES”) site in the stomach.

43. The system of claim 41, further comprising an endoscope for locating the desired tissue site from within the stomach.

44. A viewing lens for use with an endoscope or laparoscope comprising;
a body having a proximal end and a distal end;
an attachment portion coupled to the proximal end configured to sealingly attach to the distal end of an endoscope or a laparoscope; and
a clear lens portion coupled to the distal end; wherein a distance between the clear lens and attachment portion is compatible with a focal length of the endoscope or laparoscope to allow tissue viewed in the clear lens to be in focus.

45. The lens of claim 44, further comprising a port configured to communicate directly with an endoscope or laparoscope channel.

46. The lens of claim 44, wherein the lens is made of a plastic, glass, or combination of the two materials.

47. The lens of claim 44, wherein the lens comprises a spherical tip to prevent trauma to surrounding organs during use.

48. The lens of claim 44, wherein a portion of the viewing lens and/or sides are opaque to enhance visibility within the peritoneal cavity.

49. The lens of claim 44, wherein the body has outward tapered sides along its length from the attachment portion to the lens.

50. The lens of claim 44, wherein the body may be made of pliable optically clear materials such as polyurethane or silicone.

51. The lens of claim 44, wherein the body includes an inflatable portion configured to displace organs within the peritoneal cavity for the purpose of creating space within the peritoneal cavity with limited insufflation.

52. A method for positioning a guide wire between a patient's mouth and a skin site via a desired tissue site in a stomach, the method comprising:
creating a working port into the stomach from an access port at the skin site;
advancing an end of a guide wire through the access port and working port into the stomach;
creating an implant tract at the desired tissue site;
advancing a handleless forceps into the stomach and through the implant tract into the peritoneal cavity;
grasping the guide wire within the stomach with an endoscopic grasper and pushing the guide wire back in to the peritoneum through the working port;
steering the endoscopic grasper and guide wire toward the forceps;
grabbing the guide wire with the forceps;
withdrawing the handleless forceps and guide wire from the mouth; wherein the guide wire extends between the mouth and the skin site via the implant tract.

53. The method of claim 52, wherein the desired tissue site is a pes anserinus (“PES”) site in the stomach.

54. The method of claim 52, further comprising sealing the working port.

55. The method of claim 52, wherein the access port and working port are made using a percutaneous endoscopic gastrostomy (PEG) procedure.
